(19) United States
(12) Patent Application Publication
(10) Pub. No.: US 2024/0077422 A1
(43) Pub. Date: Mar. 7, 2024

(54) DIAGNOSTIC MEANS FOR THE DETECTION AND/OR QUANTIFICATION OF A PLURALITY OF ANALYTES PRESENT IN A SAMPLE

(71) Applicants: Vincent Chabottaux, Wandre (BE); Thomas Glouden, Liège (BE); Benoit Granier, Neupré (BE)

(72) Inventors: Vincent Chabottaux, Wandre (BE); Thomas Glouden, Liège (BE); Benoit Granier, Neupré (BE)

(73) Assignee: UNISENSOR, Neupré (BE)

(21) Appl. No.: 18/461,044

(22) Filed: Sep. 5, 2023

Related U.S. Application Data
(62) Division of application No. 16/652,800, filed on Apr. 1, 2020, now abandoned.

(30) Foreign Application Priority Data
Oct. 4, 2017 (BE) BE2017/5705
Oct. 4, 2017 (BE) BE2017/5706
Oct. 4, 2017 (BE) BE2017/5707
Oct. 4, 2017 (BE) BE2017/5708
Oct. 4, 2017 (BE) BE2017/5709

(51) Int. Cl. G01N 21/64; G01N 21/25; G01N 33/543; G01N 33/58; G01N 33/94

(52) U.S. Cl. CPC G01N 21/6428; G01N 21/253; G01N 21/6452; G01N 21/6456; G01N 33/54388; G01N 33/582; G01N 33/9446; G01N 2021/6439; G01N 2021/7759; G01N 2201/0692

(57) ABSTRACT

Immuno-chromatographic diagnosis means (1) for detecting and/or quantifying a plurality of analytes present in an essentially liquid sample (E), comprising:
at least one reaction mixture (2) containing recognition biological molecules and/or competitive ligands labelled with at least one visualisation molecule which is detectable in fluorescence, said reaction mixture being present in a separate container of said recovery system (3); and
at least one recovery system (3) in the form of a solid support to which are bonded competitive ligands and/or recognition biological molecules at distinct and known recovery locations (4 and 5), which are arranged according to a two-dimensional matrix arrangement defined according to a system of coordinates, so as to identify by the localisation of said recovery locations (4 and 5) on said support, said analytes present in said sample (E).

### TECHNICAL FIELD

The present invention relates to an immuno-chromatographic diagnosis means for respectively, simultaneously and specifically detecting and/or quantifying a plurality of analytes present in an essentially liquid sample, comprising:
at least one reaction mixture containing recognition biological molecules and/or competitive ligands labelled with at least one visualisation molecule; and
at least one recovery system in the form of a solid support to which are bonded, competitive ligands and/or recognition biological molecules at distinct and known recovery locations, so as to identify by the localisation of said recovery locations on said support, said analytes present in said sample.

### TECHNOLOGICAL BACKGROUND

These days, the interest in diagnosis means making it possible to respectively, simultaneously and specifically detect and/or quantify analytes present in a sample is growing, and this particularly in the field of food products, likewise in the medical field.

Indeed, new problems with public health emerging must continually be coped with, against which rapid and effective diagnosis solutions must be developed in view of providing a suitable treatment. For example, each year throughout the world, around 60000 human intoxications are linked to the toxins produced by algae (including soft water cyanotoxins), with a total mortality of approximately 1.5%. Marine biotoxins (also called phycotoxins) are produced by certain phytoplankton species and are likely to be accumulated in various marine species, for example in fish, crabs or filter-feeding bivalves (shellfish) such as mussels, oysters, scallops and clams. If someone consumes significant quantities of contaminated shellfish, they can be a victim of a serious intoxication. It is therefore crucial to have rapid and effective diagnosis means to detect these marine biotoxins, for example via an analysis of the blood or urine.

Diagnosis means such as defined above can also be used for detecting and quantifying viruses responsible for lots of various pathologies. Such diagnosis means would make it possible: (1) to provide proof of the viral origin of the clinical signs observed and to diagnose the virus responsible (for example, hepatitis or herpes) and to monitor the biological evolution of the infection (for example, via the quantification of the virus in the blood: HIV, HBV, HCV); (2) to monitor a biological evolution of the infection (for example, HIV or hepatitis B); (3) to make it possible for a therapeutic decision and to judge the effectiveness of antiviral treatments (for example, for the treatment of a cytomegalovirus infection by ganciclovir); (4) to prevent the transmission of viral infections when giving blood, organs and tissues; (5) to assess the immune status (for example, in the case of rubella); (6) to study the serum markers in the population (for example, during prevalence investigations or epidemiological studies). Generally, the medical diagnosis aims for a maximum extent of the parameters to be detected to better target the treatment and the type of care to provide to the patient, which limits, in particular, the secondary effects which are often not very well-known.

Moreover, in the food sector and more specifically, in the dairy industry, monitoring and controlling products, involves carrying out tests at the earliest possible stage of the manufacture thereof. Ideally, these tests must be carried out in the place of producing raw materials or in the place of their transformation thereof. These screening tests are particularly designed to detect the presence and the quantity of certain analytes, of which are chemical contaminants (for example, antibiotic residues and toxins), proteins (for example, allergens) or pathogens (for example, viruses, parasites or bacteria). The increase of health standards and the desire for a better traceability of food products, involves an increase of analytes to be tested, as well as knowing as precisely as possible, the classes thereof (identifications of families, classes and of the specific compound) and the quantities thereof with respect to the maximum limits authorised in each of the matrices. Moreover, with milk coming from numerous, many various places throughout the world, it is difficult to determine specifically the contaminants which could be found in milk according to the place of production, as practices are different from one place in the world to another. Indeed, the origin of foodstuffs, as well as the associated local production practices are not always known, which obligates to detect as a broad spectrum of compounds, as broad as possible covering everything which can be found in the sample to be analysed.

In particular, the agribusiness is interested in a diagnosis means making it possible to consider in one single operation, the analysis of compounds belonging to different classes which could have fundamentally different physico-chemical properties, within one same family of analytes or not, and present simultaneously in a given sample. For example, the type and the number of antibiotics which can be administered to animals can vary according to a therapeutic or prophylactic application, according to the animal species, the germ to be fought against, veterinary practices, legislation in force, available means or also geographic regions. In the case of certain particular treatments, a drug mixture can be used. As a general rule, the practitioner uses, by itself or in combination of antibiotic products selected from among all the compounds commercially available according to the assessment thereof of the best effectiveness.

The main classes of antibacterial agents and antibiotics are: penicillins and cephalosporines, tetracyclines, sulphonamides, aminoglycosides and aminocyclitols, macrolides, chloramphenicols or other peptides, ionophores, nitrofuran antibiotics, quinolones, carbadox, etc., each of these classes grouping together a very vast set of chemically different compounds.

The presence of such molecules in dairy products can have a major negative impact on the profitability of the industrial method involving a fermentation (cheese, yogurt, etc.) from fresh milk.

In addition, the use, sometimes intensive, of antibiotics in veterinary medicine and in farming production could be at the origin of bacterial strains emerging which have become resistant to antibiotics. To preserve human health and legislate in this regard, numerous countries have established maximum residue limits (MRL) for antibiotic residues in foodstuffs. These MRLs bond the limit between a positive sample and a negative sample, i.e. between a refused sample and an accepted sample.

It is important that the screening methods call upon a diagnosis means (1) that can cover the simultaneous detection of a maximum of compounds, the screening tests therefore needing to preferably and logically be multi-analyte tests, (2) that can make it possible to know the classes to which the compounds found in a positive sample belong, so as to be able to directly orient towards the suitable confirmation method, and (3) that cannot give “false negative” type results, as these will subsequently avoid the analysis and will not subsequently be confirmed.

### STATE OF THE ART

A diagnosis means such as indicated at the start is known. Indeed, document EP1712914 discloses an immuno-chromatographic diagnosis means for respectively, simultaneously and specifically detecting and/or quantifying a plurality of analytes present in an essentially liquid sample, comprising:
at least one reaction mixture containing recognition biological molecules and/or competitive ligands labelled with at least one visualisation molecule; and
at least one recovery system in the form of a solid support to which are bonded, competitive ligands and/or recognition biological molecules at distinct and known recovery locations, so as to identify by localising said recovery locations on said support, said analytes present in said sample.

More specifically, this document provides a diagnosis means making it possible to simultaneously detect a set of compounds which could belong to at least two separate classes of analytes and to characterise the class to which a detected compound actually belongs, and this by demonstrating the technical and practical compatibility of combining in one single and same method, at least two detection mechanisms without the functioning of either being able to interfere with the functioning of the other. Furthermore, a diagnosis means according to document EP1712914 demonstrate the technical feasibility of a multi-analyte dosage which can be performed rapidly, for example in less than 10 minutes, and in one single and same analysis step at the start of one single and same sample.

Practically, the method for implementing the diagnosis means according to document EP1712914 is characterised by the following steps:
putting a predetermined reaction mixture in contact with a sample to be characterised to obtain a solution which is incubated at 50° C. for 3 minutes;
soaking the recovery system defined above in the solution obtained and incubation for 3 minutes;
quantitative and qualitative interpretation of the result on the recovery system by means of an optical reading device.

According to this document, it is the positioning of the recovery elements (competitive ligands) which will make it possible to identify the type of contamination. For example, according to document EP1712914 which corresponds to the simultaneous dosage of tetracyclines, β-lactams and sulphonamides, each recovery element is arranged in the form of a capture line, each of them being arranged successively behind one another by referring to the migration direction of the liquid (corresponding to the reaction mixture put into contact with a sample). According to a preferred modality of this diagnostic means, the capture zones comprising the recovery elements of the β-lactams, tetracyclines and sulphadimethoxine are arranged respectively to a first, to a second and to a third level by referring to the migration direction of the liquid.

The interpretation of the results according to such a diagnosis means must be done conversely and is based on the competition principle which exploits the recognition of the compounds sought regarding a competitive ligand and/or a recognition biological molecule. Several cases can be presented when the recovery elements bonded on the recovery system are competitive ligands:
either the compound sought is present in the sample, and will thus be linked to the recognition biological molecules present in a reaction mixture which will consequently no longer be free to be bound to the competitive molecules bonded to the recovery system. The result will thus be positive and will have a marking which is absent;
or the compound sought is absent from the sample, the recognition biological molecules present in a reaction mixture therefore being free to be bound to the competitive molecules bonded to the recovery system. The result will thus be negative and will have a marking which is present.

Unfortunately, a diagnosis means according to document EP1712914 only makes it possible to detect and/or quantify a limited number of analytes present in a sample and cannot therefore be considered as actually being a multi-analyte diagnosis means. More specifically, the diagnosis means according to document EP1712914 makes it possible to detect the compounds belonging to three separate classes of antibiotics only, namely 3-lactams, tetracyclines and sulphonamides.

Consequently, even if the β-lactams, the tetracyclines and the sulphonamides actually constitute classes of analytes which could be considered as being different, this document makes it possible to only detect antibiotics. Thus, a diagnosis means according to this document does certainly not make it possible to detect and/or quantify analytes, like antibacterial agents, toxins, hormones, pathogens, adulterants or also allergens.

Indeed, the sectors concerned such as the agribusiness and the medical sector demand an analysis which is as complete as possible, and which can preferably identify a maximum number of compounds. It is more practical and more economical to carry out one single multiple test from one single sample rather than needing to carry out a particular test for each compound or for only one small group of compounds, namely 2 or 3 compounds as a maximum, such as is the case with a diagnosis means according to document EP1712914.

As described above, this document comprises a recovery system having capture zones (bonded recovery elements) in the form of lines arranged behind one another and perpendicularly to the migration direction of the liquid, a technical incompatibility is met when a person skilled in the art attempts to arrange a greater number of capture zones simultaneously on the recovery system, and this because of (1) the restricted size of the test zone, (2) the greater quantity of reagents to be deposited on the successive lines (favouring a background noise and more significant inter-reactivities) and (3) a lack of precision during the interpretation of the results with a visual or instrumental analysis which is long and complex. It therefore becomes difficult, even impossible to delimit the different capture zones from one another and therefore to distinguish the different analytes from one another.

The publication by Taranova (Taranova et al., 2013) attempts to solve the shortcomings of document EP1712914 by combining immuno-chromatography and “microarray” technology. Indeed, in view of increasing the number of analytes which could be detected/quantified in one single test, the document by Taranova proposes arranging the recovery locations on the solid support according to a two-dimensional matrix. In this way, the solid support of the immuno-chromatographic diagnosis according to Taranova has a microarray compounds of 32 antigens (competitive ligands) bonded in the form of points (recovery locations). Unfortunately, a diagnosis means according to this document makes it possible to only detect and quantify four analytes, namely amphetamine, benzoylecgonine, methamphetamine and morphine, which are recognised as being drugs of abuse. Indeed, according to Taranova et al., eight recovery locations in the form of points are provided on the solid support for detecting and quantifying one single analyte. Specifically, for detecting and/or quantifying a given analyte, eight points comprising specific antigens (competitive ligands) of this analyte are bonded on the solid support, the eight points making it possible to identify the given analyte being arranged about the axis perpendicular to the migration direction of the liquid. Consequently, according to this document, this is 32 different antigens, which make it possible to detect 32 different analytes which are bonded on the solid support, but only four different antigens reproduced eight times which are specific from four different analytes. Thus, identifying the analytes is only done according to one single dimension, the eight recovery locations arranged about the axis perpendicular to the migration direction being identical, namely that they comprise specific antigens of one single analyte. According to this document, the arrangement of the recovery movements in the form of points is done along rows of points, each row corresponding to a given analyte, and not according to a real two-dimensional matrix arrangement.

Thus, the immuno-chromatographic diagnosis means of the state of the art meet, at this stage, a significant limit to the effectiveness which is characterised by the absence of a test which is actually multi-analyte, which is rapid and practical and which makes it possible for a detection and/or a quantification of analytes which is:
specific, i.e. which distinguishes analytes of different classes, and
universal, i.e. which is applicable for most substances which are useful to analyse in the fields of agribusiness and medical diagnosis such as drug residues (for example, antibiotics and antibacterial agents), toxins, hormones, pathogens, adulterants or also allergens.

### AIM OF THE INVENTION

The invention aims to overcome the disadvantages of the state of the art by providing a diagnosis means, which is quicker, more practical, more economical, more effective and which makes it possible for a detection and/or a quantification of analytes which is:
specific, i.e. which distinguishes analytes of different classes, and
universal, i.e. which is applicable for most substances which are useful to analyse in the fields of agribusiness and medical diagnosis such as drug residues (for example, antibiotics and antibacterial agents), toxins, hormones, pathogens, adulterants or also allergens,
   the detection and/or quantification being carried out in one single step and in less than 15 minutes.

More specifically, a diagnosis means according to the invention makes it possible to detect and/or quantify at least 5 different classes of analytes, preferably at least 10 different classes of analytes, preferably at least 15 different classes of analytes present in a sample, the classes of analytes being drug residues (for example, antibiotics or antibacterial agents), toxins, hormones, pathogens, adulterants or also allergens, and this in less than 15 minutes and in one single step. To resolve this problem, an immuno-chromatographic diagnosis means is provided, according to the invention, to respectively, simultaneously and specifically detect and/or quantify a plurality of analytes present in an essentially liquid sample, comprising:
at least one reaction mixture containing recognition biological molecules and/or competitive ligands labelled with at least one visualisation molecule; and
at least one recovery system in the form of a solid support to which are bonded, competitive ligands and/or recognition biological molecules at distinct and known recovery locations which are arranged according to a two-dimensional matrix arrangement, so as to identify by the localisation of said recovery locations on said support, said analytes present in said sample,
   said diagnosis means being characterised in that,
a) said two-dimensional matrix arrangement is defined according to a system of coordinates having a first coordinate X and a second coordinate Y, such that each recovery location bonded on said solid support makes it possible to identify a distinct analyte;
b) to detect and/or quantify a given analyte, a diagnosis couple consisting of a competitive ligand and of a recognition biological molecule is present, such that said recognition biological molecule is found in said reaction mixture and said competitive ligand is bonded at at least one recovery location, or conversely;
c) said at least one visualisation molecule is a molecule which is detectable in fluorescence; and
d) said reaction mixture is present in a container, said container being separate from said recovery system.

By the term “analyte”, this means, in the sense of the present invention, a compound which constitutes an interest in being detected and/or quantified to provide a diagnosis, particularly in the agribusiness and medical field.

By the terms “class of analytes”, this means, in the sense of the present invention, a grouping together of several analytes which have similar biological and chemical properties. As an example, the drug residues can be separated into different classes, such as penicillins, cephalosporines, tetracyclines, sulphonamides, aminoglycosides, aminocyclitols, macrolides, quinolones, ionophores, carbadox, nitrofuran antibiotics and phenicols. Specifically, penicillins are antibiotics which have a common action mode (biological property) and which have a similar chemical structure (chemical property).

By the terms “respective detection and/or quantification”, this means, in the sense of the present invention, a detection and/or a quantification of all the analytes of interest by using one single diagnosis means according to the invention.

By the terms “simultaneous detection and/or quantification”, this means, in the sense of the present invention, a detection and/or a quantification of all the analytes of interest after an identical time lapse.

By the terms “specific detection and/or quantification”, this means, in the sense of the present invention, a detection and/or a quantification of all the analytes of interest separately, so as to be able to precisely identify the analyte which is detected and/or quantified.

By the terms “diagnosis couple”, this means, in the sense of the present invention, two complementary molecules intended to detect and/or quantify a given analyte, said two molecules being a recognition biological molecule and a competitive ligand. The detection and/or the quantification of the given analyte is based on the principle of competition according to two possible situations:
either the recognition biological molecule is found in the reaction mixture and the complementary competitive ligand is bonded on the solid support;
or the competitive ligand is found in the reaction mixture and the complementary recognition biological molecule is bonded on the solid support.

By the terms “recognition biological molecules”, this means, in the sense of the present invention, a natural or synthetic molecule which is capable of being bound specifically to an analyte of interest.

By the terms “competitive ligands”, this means, in the sense of the present invention, a molecule which is capable of being bound specifically to the recognition biological molecules and which will therefore enter into competition with an analyte of interest for the binding to the recognition biological molecules.

By the terms “recovery location”, this means, in the sense of the present invention, a placement at which recognition biological molecules or competitive ligands will be bonded. In the case where recognition biological molecules are bonded at a recovery location, the analyte of interest (if it is present) or the competitive ligand (if the analyte of interest is absent) will be bound specifically, be captured and therefore stop migrating. In the case where competitive ligands are bonded at a recovery location, the specific recognition biological molecules of an analyte of interest, will be bound specifically, be recovered and therefore stop migrating, and this if the analyte of interest in absent.

The method implemented for the detection and/or the quantification is as follows:
contacting the reaction mixture with the sample to obtain a liquid;
incubating at a temperature of 30° C. for 3 minutes;
soaking the end of the recovery system which is found upstream of the migration direction in the liquid (comprising the sample and the reaction mixture);
incubating for 10 minutes at 30° C.; and
interpreting qualitatively and/or quantitatively the result on the recovery system by means of an optical reading device.
   The migration direction of the liquid according to the invention is defined according to said system of coordinates defining the matrix arrangement of the recovery locations bonded on the recovery system according to the invention and is done consequently according to a coordinate X and a coordinate Y.

The detection and/or the quantification according to the invention is based on the principle of competition which exploits the recognition of the analytes sought regarding a competitive ligand and/or a recognition biological molecule.

Several cases can be presented according to competitive ligands or recognition biological molecules are bonded to the recovery locations:
1) In the case where the recovery elements are competitive ligands,
   either the compound sought is present in the sample, and will thus be bound to the recognition biological molecules present in a reaction mixture which will consequently not be free to be bound to the competitive molecules bonded to the recovery system. The result will thus be positive and will have a marking which is absent;
   or the compound sought is absent from the sample, the recognition biological molecules present in a reaction mixture therefore being free to be bound to the competitive molecules bonded to the recovery system. The result will thus be negative and will have a marking which is present.
2) In the case where the recovery elements are recognition biological molecules,
   either the compound sought is present in the sample, and will thus enter into competition with the competitive ligands present in a reaction mixture to be bound to the recognition biological molecules bonded to the recovery system. The result will thus be positive and will have a marking which is absent or weak;
   or the compound sought is absent from the sample, the competitive ligands thus being the only to be bound to the recognition biological molecules bonded to the recovery system. The result will thus be negative and will have a marking which is present.

In the scope of the present invention, it has surprisingly been demonstrated that an immuno-chromatographic diagnosis means which comprises the features (a), (b) (c) and (d) such as indicated above, mean that the diagnosis means according to the invention is more effective and is both specific and universal. Specifically, it makes it possible to detect and/or quantify at least 5 different classes of analytes, preferably at least 10 different classes of analytes, preferably at least 15 different analytes present in a sample, the classes of analytes being drug residues (for example, antibiotics or antibacterial agents), toxins, hormones, pathogens, adulterants or also allergens, and this in less than 15 minutes and in one single step. A diagnosis means according to the invention is consequently more practical, more economical and more effective than the diagnosis means currently known which are limited to the detection and/or quantification of less than five different classes of analytes.

Specifically, it has surprisingly been observed that the placement of the reaction mixture in a container separated from the solid support brought several advantages.

First, with the interaction of the reaction mixture with the sample analysed being focalized in a separate container, the control of the interaction of the reaction mixture with the sample is optimised. By doing so, it is certain that the sample which interacts completely with the reaction mixture before the liquid thus obtained (formed from the reaction mixture and from the sample) is not in contact with the solid support and therefore the recovery locations. Consequently, the separation of the reaction mixture in a container separated from the solid support makes it possible to avoid obtaining false negatives. Indeed, in the case where the reaction mixture is bonded on the solid support upstream of the recovery elements with respect to the migration direction as is the case in the document by Taranova et al., the mainly liquid sample is directly put into contact with the reaction mixture bonded on the solid support, which will lead to the immediate migration of the liquid by capillarity. The risk is thus increased that the sample meets the recovery locations before the interaction with the reaction mixture is complete, leading to an erroneous result, i.e. that the analyte is actually present in the sample, but that it is not detected.

Second, the separation of the reaction mixture in a container makes it possible to have a better control of the sample quantity which is analysed. Indeed, with a diagnosis means according to the invention, it is possible to deposit a defined and specific sample volume in the container and to ensure that all of this sample volume will be analysed, and this contrary to the diagnosis means disclosed by Taranova et al. Indeed, with such a diagnosis means, i.e. where the reaction mixture is present on the solid support upstream of the recovery elements with respect to the migration direction of the liquid, the sample is directly put into contact with the solid support which is immersed in the sample, the liquid obtained (formed from the sample and from the reaction mixture) instantaneously migrating by capillarity. In this way, it is impossible to specifically define the liquid volume which will migrate on the solid support, which makes it very difficult, even impossible to determine the sample volume which is actually analysed. This distinctive feature of the diagnosis means according to the invention makes it possible to reduce the standard deviations and to thus obtain an improved reproducibility with respect to the diagnosis means of the state of the art. The specific determination of the sample volume, which is analysed also makes it possible to define, more suitable, the composition of the reaction mixture and particularly, of the quantity of the different elements which compose it. Consequently, the detection or the quantification of a given analyte is significant and weak, even in one single sample, and this contrary to the document by Taranova. Indeed, according to the document and as cited above, eight recovery locations must be provided on the solid support for detecting and quantifying one single analyte. Therefore, for the weak detection and/or quantification of one same number of analytes, for example four analytes, a solid support according to the invention must comprise 4 recovery locations, while a solid support according to Taranova et al. must comprise 32 recovery locations. Consequently, for one same solid support surface, the diagnosis means according to the invention makes it possible to detect and/or to quantify 32 different analytes.

Third, the separation of the reaction mixture in a container makes it possible to increase the number of components of the reaction mixture. Indeed, as described above, to detect and/or quantify a given analyte, a pair of diagnoses constituted of a competitive ligand and of a recognition biological molecule is present, such that the recognition biological molecule is found in the reaction mixture and the competitive ligand is bonded in at least one recovery placement, or conversely. Therefore, the reaction mixture contains one recognition molecule or one competitive ligand per analyte. Consequently, to detect and/or quantify a large number of different analytes, a greater number of different recognition molecules or competitive ligands must be added in the reaction mixture. With the diagnosis means of the state of the art, as disclosed by Taranova et al., the number of components of the reaction mixture is limited by the surface available on the solid support.

Furthermore, according to the invention, the two-dimensional matrix arrangement is defined according to a system of coordinates having a first coordinate Xand a second coordinate Y, such that each recovery location bonded on said solid support makes it possible to identify a separate analyte. This feature also significantly improves the effectiveness of the diagnosis means according to the invention by providing a diagnosis means which makes it possible to detect and/or quantify a greater number of separate analytes for an identical support surface, for example to improve the effectiveness by eight times with respect to the diagnosis means according to Taranova et al.

Thus, according to the invention, for one same coordinate X, several recovery locations, each comprising different recognition biological molecules or competitive ligands, are arranged along different coordinates Y thus making it possible to detect and/or to quantify, for one same coordinate X, several different analytes. Conversely, for one same coordinate Y, several recovery locations, each comprising different recognition biological molecules or competitive ligands, are arranged along different coordinates X, thus making it possible to detect and/or to quantify several different analytes.

Moreover, it has surprisingly been observed that the use of a visualisation molecule which is detectable in fluorescence makes it possible to improve the detection limit of the signal, and therefore to reduce the risk of false negatives by increasing the sensitivity of the detection and/or of the quantification. Consequently, with the detection threshold being lower, the recovery locations can be smaller, which makes it possible to obtain a solid support which comprises more recovery locations for an identical surface. Moreover, with the detection of the signal by fluorescence being more sensitive, a lower quantity of competitive ligands and/or of recognition biological molecules must be bonded to the recovery locations, which gives a significant economic advantage, but also a background noise which is reduced and a risk of inter-reactions between the detection and/or quantification mechanisms which is decreased.

In conclusion, a diagnosis means according to the invention provides a greater technical effect with respect to current diagnosis means and more specifically, with respect to the diagnosis means according to the document by Taranova et al.

Thus, the present invention demonstrate the technical and practical compatibility of combining in one single and same detection means, an increased number (at least 5, preferably at least 10, preferably at least 15) of detection and/or quantification mechanisms. A technical feasibility has moreover been highlighted, in the scope of the present invention, of a multi-analyte dosage which can be performed quickly, in less than 15 minutes, preferably in 13 minutes, and in one single and same analysis step using one single and same sample. Indeed, the diagnosis means according to the invention does not require any scrubbing nor producing a separate step of marking recognition molecules and/or competitive ligands with at least one visualisation molecule being given that, according to the invention, the reaction mixture comprises recognition molecules and/or competitive ligands coupled with at least one visualisation molecule.

Preferably, said recovery locations bonded on said recovery system of said diagnosis means according to the invention, are arranged according to a two-dimensional matrix arrangement in the form of points, each having a diameter of between 20 μm and 2 mm, preferably of between 100 μm and 500 μm, preferably between 250 μm and 400 μm.

It has been demonstrated that recovery locations in the form of points, each having a diameter of between 20 μm and 2 mm, preferably of between 100 μm and 500 μm, preferably between 250 μm and 400 μm, made it possible to bond at least 5, preferably at least 10, preferably at least 15 recovery locations in one single sample, in two samples or in three samples for detecting and/or quantifying at least 5, preferably at least 10, preferably at least 15 different analytes, as well as at least one recovery placement deposited in one single sample, in two samples or in three samples, intended for controlling the detection threshold making it possible to validate the test and/or the calibration for the detection and/or the quantification, and this on a recovery system having a reasonable size, to carry out the detection and/or the quantification of said at least 15 analytes by an optical reading device.

Advantageously, the recovery locations bonded on said recovery system of said diagnosis means according to the invention are arranged according to a two-dimensional matrix arrangement in the form of points, said points being present at a density of between 62500 and 6.25 points per cm², preferably of between 2500 and 100 points per cm², preferably of between 400 and 150 points per cm².

Preferably, the matrix arrangement of all the recovery locations is less than or equal to 3 cm², preferably less than or equal to 2 cm², preferably less than or equal to 1 cm².

Advantageously, the first coordinate X is defined on a longitudinal axis of a length of said recovery system and the second coordinate Y is defined on a longitudinal axis of a width of said recovery system.

It is reasonable to provide a minimum space distance between two points which is of between 20 μm and 2 mm, preferably between 100 μm and 500 μm, preferably between 250 μm and 400 μm, according to the coordinates X and Y.

In a particular embodiment, said recovery system according to the invention comprises at least 5, preferably at least 10, preferably at least 15 separate recovery locations intended to respectively, simultaneously and specifically detect and/or quantify at least 5, preferably at least 10, preferably at least 15 separate analytes present in a sample, and at least one recovery placement intended for a control and/or a calibrator.

Preferably, said control and/or said calibrator is obtained from an independent competitive ligand/recognition molecule pair, of which the intrinsic (or synthetic) nature means that the control molecule is never present in the sample (for example, a specific antibody of a protein or another animal species different from that of which the sample comes) or a carrier protein (for example, bovine serum albumin) chemically modified with a synthetic marker (for example, a biotin or a poly-histidine or c-Myc marker).

In a particularly advantageous embodiment of the diagnosis means according to the invention, each of said recovery locations is arranged on said recovery system in duplicate, preferably in triplicate. Performing duplicates or triplicates makes it possible to also improve the statistic and the precision of the results obtained.

Preferably, the matrix arrangement of the recovery locations to which competitive ligands or recognition molecules are bonded, is determined by the migration direction of the liquid, such that a recovery placement to which competitive ligands or recognition biological molecules are bonded, intended to detect and/or quantify a first given analyte is localised upstream of a recovery placement to which competitive ligands or recognition biological molecules are bonded, intended to detect and/or quantify a second given analyte, and this with respect to the migration direction of the liquid. Such a matrix arrangement also makes it possible to decrease the risk of inter-reactions between the different mechanisms for detecting and/or for quantifying analytes of interest.

Advantageously, said recovery system in the form of a solid support comprises a membrane or a set of membranes. Preferably, the membrane is a nitrocellulose membrane.

Advantageously, said container is a glass or plastic container.

Advantageously, said recognition biological molecules are antibodies, preferably primary antibodies, either monoclonal or polyclonal, purified or non-purified, and/or aptamers and/or GEPIs and/or biological receptors.

Advantageously, said competitive ligands are similarto the analytes sought and/or molecules capable of specifically bonding said recognition biological molecules.

In a particularly advantageous embodiment of the diagnosis means according to the invention, said competitive ligands are selected from the group constituted of drug substances of antibiotic, hormone, toxin type such as Aflatoxin, viruses of the Dengue type, L-type bacteria, monocytogenes, heavy metals, adulterants, allergens, and the mixtures thereof.

Preferably, said at least one visualisation molecule is fused with said recognition biological molecules and/or with said competitive ligands via a chemical and/or genetic coupling.

By the terms “chemical and/or genetic coupling”, it is understood in the sense of the present invention, a bonding of the recognition molecule and/or the competitive ligand to the visualisation molecule via a chemical and/or genetic modification of the recognition biological molecule and/or of the competitive ligand, these consequently no longer being in the natural state thereof but in a modified form or in a complex form.

It has been demonstrated that such a chemical and/or genetic coupling of the visualisation molecule to the recognition biological molecules and/or to the competitive ligands present in the reaction mixture makes it possible to also improve the technical and practical compatibility of combining in one single and same means for detecting and/or quantifying an increased number (at least 5, preferably at least 10, preferably at least 15) of detection and/or quantification mechanisms without the functioning of one of them being able to interfere with the functioning of one of the other mechanisms. Indeed, a reaction mixture according to the invention which has such a coupling offers the advantage of decreasing, even removing the risk of a specificity and of inter-reactions between the different recognition biological molecules and/or the different competitive ligands present in the reaction mixture, and thus the risk of observing false positives and/or false negatives, but also of considerably decreasing the residual marking (background noise) observed on the recovery system when such a coupling is not present and of thus obtaining a better contrast between the marking of the recovery elements and of the non-bonded solid support (and of thus obtaining a better detection threshold).

Document EP1712914 recommends, on the contrary, that no marking by chemical modification takes place in order to preserve, to the maximum, the functionalities of the receptors and antibodies used, and that consequently, the recognition biological molecule s are exploited in the most natural state as possible thereof. For example, a recognition biological molecule like a receptor is labelled using an antibody, themselves recognised by a protein A (recognising all the types of antibodies, generally) which is conjugated with colloidal gold. According to this document, it is therefore the protein A, and not the recognition biological molecule, which is coupled with colloidal gold (the visualisation molecule).

Advantageously, said chemical and/or genetic coupling is achieved via at least one electrostatic force, at least one peptide bond, at least one reporter gene, or a combination thereof.

In a particular embodiment, said at least one visualisation molecule is selected from the group constituted of fluorescein isothiocyanate (FITC), phycoerythrin (PE), rhodamine B and the mixtures thereof.

Preferably, said analytes are selected from the group consisting of drug residues, toxins, viruses, bacteria, hormones, heavy metals, adulterants, allergens and the mixtures thereof. From among the drug residues, in particular antibiotics and antibacterial agents are found. Undesirable chemical molecules, adulterants, can also be detected following a passive contamination by transfer of the container (for example, from a plastic packaging).

In a particular advantageous embodiment, said analytes are drug residues and are selected from the group constituted of penicillins, cephalosporines, tetracyclines, sulphonamides, aminoglycosides, aminocyclitols, macrolides, quinolones, ionophores, carbadox, nitrofuran antibiotics, phenicols, and the mixtures thereof.

Advantageously, said sample is obtained from milk, honey, meat, eggs, whole blood, serum, urine, or other biological liquids.

By the terms “biological liquids”, this means, in the sense of the present invention, any organic or bodily fluid liquid produced by a living organism.

Preferably, said sample is obtained from milk. It has been observed that the detection of analytes is more sensitive, when the sample analysed is obtained from milk, and this as the components of the milk saturate the nitrocellulose membrane and thus decreases the background noise.

Specifically, according to the invention, respectively, simultaneously and specifically detecting and/or quantifying a plurality of analytes present in a sample is carried out by means of an optical reading device.

In a particular embodiment according to the invention, the recovery locations are arranged according to a three-dimensional matrix arrangement. Such a three-dimensional matrix arrangement makes it possible to be arranged between a greater number of recovery locations on a solid support having a similar surface and therefore to detect and/or quantify a greater number of analytes of interest, respectively and simultaneously.

Advantageously, the three-dimensional matrix arrangement is defined according to a system of coordinates having a first coordinate (X) defined on a longitudinal axis of a length of said recovery system, a second coordinate (Y) defined on a longitudinal axis of a width of said recovery system and a third coordinate (Z) defined on a longitudinal axis of a depth of said recovery system. In this case, the migration direction of the liquid (comprising the sample and the reaction mixture) is defined according to said system of coordinates defining the matrix arrangement of the recovery locations bonded on the recovery system according to the invention and is done consequently according to a coordinate X, a coordinate Y and a coordinate Z.

Other embodiments of the diagnosis means according to the invention are indicated in the appended claims.

The invention is also based on a method for respectively, simultaneously and specifically detecting and/or quantifying a plurality of analytes present in an essentially liquid sample comprising the following steps:
contacting a reaction mixture of a diagnosis means according to the invention with the sample to obtain a liquid;
incubating at a temperature of between 0 and 70° C., preferably between 10 and 60° C., preferably between 20 and 50° C., preferably between 20 and 40° C., preferably between 25 and 35° C., preferably 30° C., for a duration less than or equal to 15 minutes, preferably less than or equal to 10 minutes, preferably less than or equal to 5 minutes, preferably less than or equal to 3 minutes, preferably equal to 3 minutes;
soaking an end of a recovery system of a diagnosis means according to the invention in the liquid;
incubating at a temperature of between 0 and 70° C., preferably between 10 and 60° C., preferably between 20 and 50° C., preferably between 20 and 40° C., preferably between 25 and 35° C., preferably 30° C., for a duration less than or equal to 15 minutes, preferably less than or equal to 10 minutes, preferably equal to 10 minutes; and
interpreting qualitatively and/or quantitatively the result on the recovery system by means of an optical device.

The method according to the invention is based on microfluidic and immuno-chromatographic technologies.

The invention also aims for a diagnosis set for respectively, simultaneously and specifically detecting and/or quantifying analytes present in a sample comprising a diagnosis means according to the invention, and further comprises a device for optically reading a removable solid support, comprising:
a placement to receive said solid support;
an optical unit to analyse said solid support and comprising:
   a first light source to emit according to an emission intensity and in a first wavelength range, a first light beam to said placement;
   an imaging system comprising an optical detector to provide an image of a visualisation zone, said visualisation zone comprising at least one portion of said placement;
   a filter to filter a defined wavelength range defined, and positioned between the placement and said imaging system;
communication means to obtain an item of information relative to a solid support;
selection means to:
   select from a list of predefined analytes corresponding to said recovery locations bonded on the solid support, a selection of analytes to be detected and/or to be quantified for said sample from one same solid support;
image processing means of said image to:
   determine, from the information relating to said solid support to be read, a finite number of subassemblies of said image, each subassembly corresponding to an analyte;
   provide data relating to light intensities coming from said subassemblies;
determination means to:
   calculate, for each subassembly corresponding to an analyte selected in said selection of analytes, a subassembly intensity;
   determine, based on said subassembly intensity, analyte information from said sample for each subassembly corresponding to an analyte selected in said selection of analytes;
transmission means configured to transmit said analyte information from said sample analysed for each subassembly corresponding to an analyte selected in said selection of analytes.

Such a device according to the invention makes it possible to read zones to be tested, in particular with an instantaneous fluorescence measurement or preferably, with a measurement of the light reflected from the zones to be tested. In order to make it possible for a selection of analytes to be tested corresponding to zones of interest on a stick, such a device proposes, thanks to the access to a method containing information relating to a stick, a selection from a list of analytes to be tested. Indeed, it is useful to select analytes to be tested before obtaining from them, the results in order to correctly target the analytes, of which it is necessary to know the results of a test in order to not expose a user to a quantity of results that is too large. Giving access to a quantity of results that is too large to a user not necessarily needing to be exposed to the risk of a loss of objectivity with respect to the initial analysis intention thereof. Thus, such a device of the invention, thanks to selection means, makes it possible to select analytes to be tested by the user before reading the stick. The selection means, in communication with the image processing means make it possible for the image processing means, to determine information from the selected analytes only.

An advantage of using the optical reader of the invention to carry out a diagnosis containing a selection of analytes of interest is that it does not require a first selection of different types of strips to be tested, nor the putting into contact of each of these strips with the product to be tested, then the positioning thereof in the optical reader. All this makes it possible to avoid a significant handling of the strips to be tested, expensive over time and stock management. This also makes it possible for a simpler, quicker and more targeted analysis of the analytes to be tested, by only having results selected from the reading of the strip by the optical reader of the invention.

Advantageously, the selection of analytes is a selection of several analytes.

Preferably, the optical device further comprises:
means making it possible to read a selection profile; and
   said selection means are configured to carry out said selection of analytes based on said selection profile.

Advantageously, each subassembly of said finite number of subassemblies of said image determined by the image processing means corresponds to said selection of analytes, preferably to each analyte selected.

Preferably, said image processing means are configured to furthermore determine said finite number of subassemblies of said image from said selection profile.

Advantageously, said first light source is configured to directly emit said first light beam directly to said placement, preferably directly to said recovery locations bonded on the solid support.

Preferably, said solid support comprises recovery locations in the form of points, each having a diameter of between 20 μm and 2 mm, preferably of between 100 μm and 500 μm, preferably between 250 μm and 400 μm.

The invention is also based on a diagnosis set for respectively, simultaneously and specifically detecting and/or quantifying analytes present in a sample comprising a diagnosis means according to the invention and further comprises an optical reading device of a removable solid support, comprising:
a placement to receive said solid support;
an optical unit to analyse said solid support and comprising:
   a first light source to emit according to an emission intensity and in a first wavelength range, a first light beam to said placement;
   a light intensity sensor to measure the emission intensity emitted by said first light source;
   a feedback means to modulate said emission intensity of said first light source according to the emission intensity measured by said light intensity sensor such that said first light source emits a target intensity;
   an imaging system comprising an optical detector to provide an image of a visualisation zone, said visualisation zone comprising at least one portion of said placement;
   a filter to filter a defined wavelength range, and positioned between the placement and said imaging system;
image processing means of said image to:
   determine a finite number of subassemblies of said image,
   provide data relating to light intensities coming from said subassemblies;
determination means to:
   calculate, for each subassembly, a subassembly intensity, and
transmission means to transmit an item of information relating to said subassembly intensity for each subassembly.

Such an optical device according to the invention makes it possible to read zones to be tested, in particular with an instantaneous fluorescence measurement or preferably, with a measurement of the light reflected from the zones to be tested. When a fluorescence technique or a reflected light technique is used to read the zones to be tests (or points), a feedback means makes it possible to guarantee an always equal excitation light intensity, which makes it possible for a reliable instantaneous fluorescence measurement or reflection, whatever the temperature, the energy source used or also the duration of use and the ageing of the light source. The use of the feedback means makes it possible to guarantee a light source having a constant intensity over time and predefined. A light energy source having a predefined intensity makes it possible, in particular, to guarantee reliable quantitative results. The feedback means is preferably an electronic feedback means. For example, the light intensity sensor is a photodiode.

The invention also aims for a diagnosis set for respectively, simultaneously and specifically detecting and/or quantifying analytes present in a sample comprising a diagnosis unit according to the invention and further comprises a device for optically reading a removable solid support, comprising:
a placement to receive said solid support;
an optical unit to analyse said solid support and comprising:
   a first light source to emit, according to an emission intensity and in a first wavelength range, a first light beam to said placement;
   an imaging system comprising a two-dimensional optical detector to provide a two-dimensional image of a visualisation zone, said visualisation zone comprising at least one portion of said placement;
   a filter to filter a defined wavelength range, and positioned between the placement and said imaging system;
image processing means of said two-dimensional image to:
   detect reference zones of said two-dimensional image,
   determine a finite number of subassemblies of said two-dimensional image,
   position in said two-dimensional image, each subassembly at a predetermined position with respect to said reference zones,
   provide data relating to light intensities coming from said subassemblies;
determination means to:
   calculate, for each subassembly, a subassembly intensity, and
transmission means configured to transmit said subassembly intensity for each subassembly.

Such an optical device according to the invention makes it possible to simultaneously read a large number of dots thanks to a two-dimensional optical detector and to image processing and determination means, making it possible to read analyte information for each of the dots. The two-dimensional image comprises subassemblies, portions, regions of interest, zones of interest or also image portions. Preferably, the subassemblies of a two-dimensional image comprise a plurality of pixels. Preferably, each subassembly comprises at least 20 pixels, preferably more than 50 pixels and also more preferably, more than 200 pixels.

The advantage of such a device according to the invention is to be able to carry out a diagnosis by a continuous fluorescence reading by avoiding a maximum background noise caused by the light source.

Another advantage of such an optical reading device of the invention is to make it possible to optically read a large number of regions of interest present on one single and same stick. In the case of such an optical device according to the invention, the reading of a large number of regions of interest does not require providing placements for several sticks. Using several sticks in one same optical reader for a simultaneous reading of several sticks in order to cover a large number of regions of interest with one same optical sensor being a source of incorrect placement and of offsetting of regions of interest from one measurement to another and this, for each of the sticks inserted in the optical reader.

The invention is also based on a diagnosis set for respectively, simultaneously and specifically detecting and/or quantifying analytes present in a sample comprising a diagnosis means according to the invention and further comprising a device for optically reading a removable solid support, comprising:
a placement to receive said solid support;
an identification device to identify a solid support to be read;
communication means to access a database of methods relating to said solid support to be read to obtain an item of information relating to a solid support;
an optical unit, to analyse said solid support based on analysis parameters comprised in said information relating to a solid support and comprising:
   a first light source to emit, according to an emission intensity, and in a first wavelength range, a first light beam to said placement;
   an imaging system comprising an optical detector to provide an image of a visualisation zone, said visualisation zone comprising at least one portion of said placement;
   a filter to filter a defined wavelength range, and positioned between the placement and said imaging system;
image processing means of said image to:
   read in the information relating to said solid support to be read, an item of information relating to a finite number of subassemblies of said image;
   provide data relating to light intensities coming from said subassemblies;
determination means to:
   calculate, for each subassembly, a subassembly intensity, and
   determine based on said subassembly intensity and based on the information relating to said solid support to be read, analyte information for each subassembly;
transmission means configured to transmit said analyte information for each subassembly.

The optical reading device according to the invention makes it possible to optically read a stick for the analysis of a sample with a selection of automated reading method. The reading method preferably comprising data relating to: a method version, a batch number, a use-by date, a type of light source used, the type of interest zone (line or point), method for qualitative (binary) or quantitative analysis, image acquisition parameters (exposure time, gain, etc.), the positions with respect to reference points (for example, according to Cartesian coordinates), a number of zones of interest, a number of replicas per analyte, the matrix organisation of zones of interest on the mobile solid support, the dimensions of the zones of interest (for example, a radius), a dimension relating to a zone around a zone of interest to be considered for considering the background, calibration parameters of the data interpolation type or making it possible for a quantitative analysis of a sample and finally, the designation of the zones of interest according to the analyte that they make it possible to detect and/or to quantify.

Other embodiments of the diagnosis set according to the invention are indicated in the appended claims.

The invention also aims for a use of a diagnosis means according to the invention for respectively, simultaneously and specifically detecting and/or quantifying analytes present in a sample, preferably at least 5, preferably at least 10, preferably at least 15 different analytes.

The invention also aims for a use of a diagnosis set according to the invention, for respectively, simultaneously and specifically detecting and/or quantifying analytes present in a sample, preferably at least 5, preferably at least 10, preferably at least 15 different analytes.

Other embodiments of use of the diagnosis means and the diagnosis set according to the invention are indicated in the appended claims.

Other features, details and advantages of the invention will emerge from the description given below, in a non-limiting manner and by making reference to the appended drawings.

### DESCRIPTION OF THE FIGURES

FIG. 1a is a schematic view of a diagnosis means according to document EP1712914.

FIG. 1b is a schematic view of a diagnosis means according to the document by Taranova et al.

FIG. 2 is a schematic view of a diagnosis means according to the invention.

FIG. 3 is a schematic view illustrating in detail, a recovery system according to the invention.

In the figures, identical or similar elements have the same references.

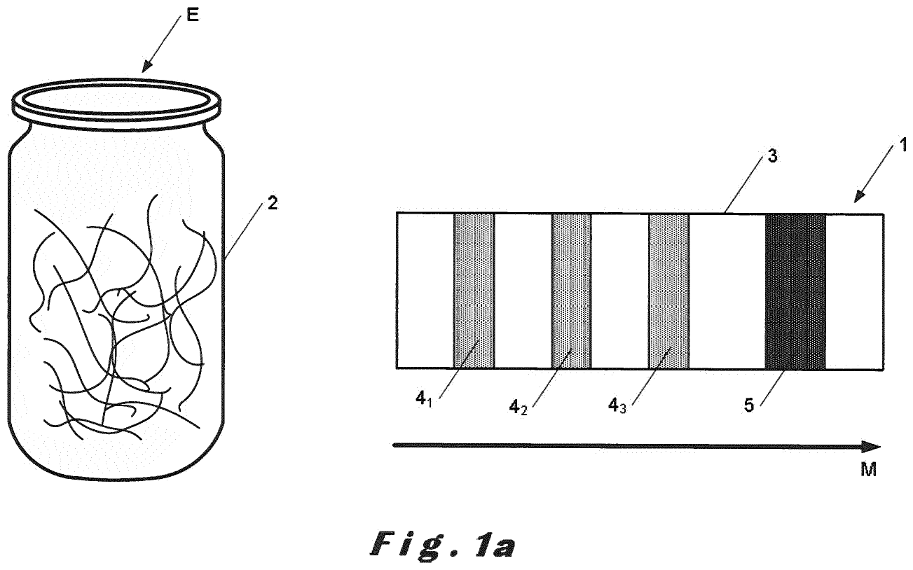

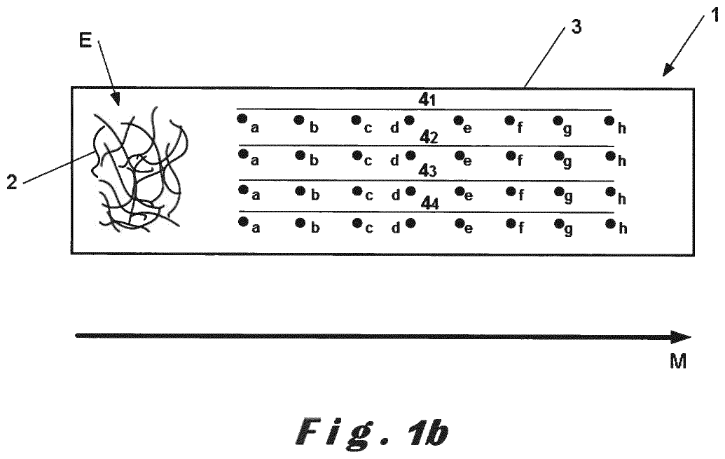

FIG. 1a represents a diagnosis means 1 according to document EP1712914 and illustrates the positioning of the recovery elements 4₁, 4₂, 4₃ and 5 on a recovery system 3 in the form of a nitrocellulose solid support in the case of the simultaneous dosage of β-lactams 4₁, tetracyclines 4₂ and sulphadimethoxine 4₃, a bonded control zone 5 also being provided, with respect to a migration direction M. According to this document, the reaction mixture 2 is provided in a separate container with which a sample E to be tested in put into contact.

FIG. 1b represents a diagnosis means 1 according to the document by Taranova et al. and illustrates the positioning of the recovery elements 4₁ₐ, 4_{1b}, 4_{1c}, 4_{1d}, 4₁ₑ, 4_{1f}, 4_{1g}, 4₁ₕ, 4₂ₐ, 4_{2b}, 4_{2c}, 4_{2d}, 4₂ₑ, 4_{2f}, 4_{2g}, 4₂ₕ, 4₃ₐ, 4_{3b}, 4_{3c}, 4_{3d}, 4₃ₑ, 4_{3f}, 4_{3g}, 4₃ₕ, 4₄ₐ, 4_{4b}, 4_{4c}, 4_{4d}, 4₄ₑ, 4_{4f}, 4_{4g}, 4₄ₕ, on a recovery system 3 in the form of a nitrocellulose solid support in the case of the simultaneous dosage of amphetamines (4₁ₐ, 4_{1b}, 4_{1c}, 4_{1d}, 4₁ₑ, 4_{1f}, 4_{1g}, 4₁ₕ), of benzoylecgonine (4₂ₐ, 4_{2b}, 4_{2c}, 4_{2d}, 4₂ₑ, 4_{2f}, 4_{2g}, 4₂ₕ), of methamphetamines (4₃ₐ, 4_{3b}, 4_{3c}, 4_{3d}, 4₃ₑ, 4_{3f}, 4_{3g}, 4₃ₕ) and of morphine (4₄ₐ, 4_{4b}, 4_{4c}, 4_{4d}, 4₄ₑ, 4_{4f}, 4_{4g}, 4₄ₕ). The recovery elements 4 are bonded in the form of points according to a two-dimensional matrix arrangement. According to Taranova et al., the reaction mixture 2 is present on said recovery system 3, in a lyophilised form, upstream of said recovery elements 4 bonded on said recovery system 3 with respect to a migration direction M of a liquid comprising the sample E to be tested on the reaction mixture 2. According to this document, the recovery elements arranged on one same row, namely having the same coordinate Y, are specific of the same analyte.

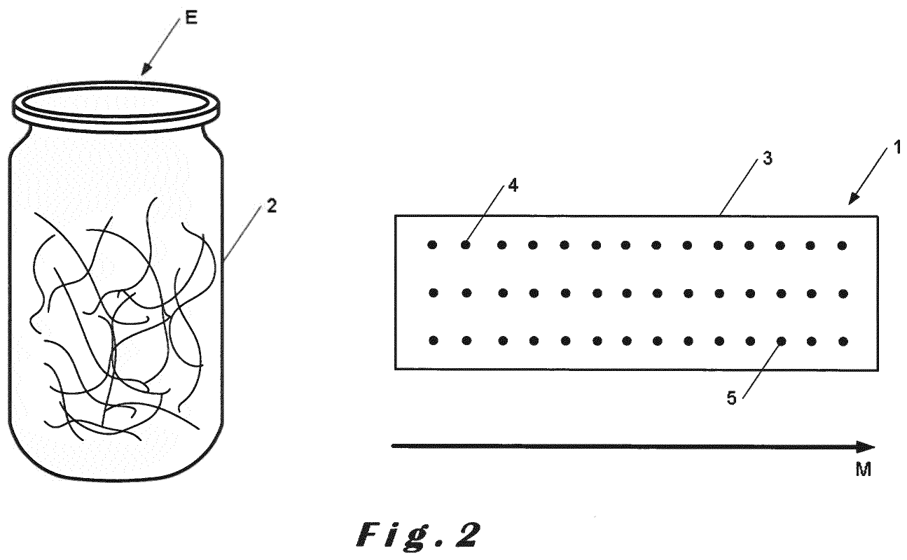

FIG. 2 represents a diagnosis means 1 according to the invention and illustrates the positioning of the recovery elements 4 and 5 on a recovery system 3 in the form of a solid support with respect to a migration direction M, the recovery elements 4 and 5 being bonded in the form of points according to a two-dimensional matrix arrangement. According to the invention, the reaction mixture 2 is provided in a separate container with which a sample E to be tested is put into contact to obtain a liquid, before soaking the recovery system 3 in the liquid obtained.

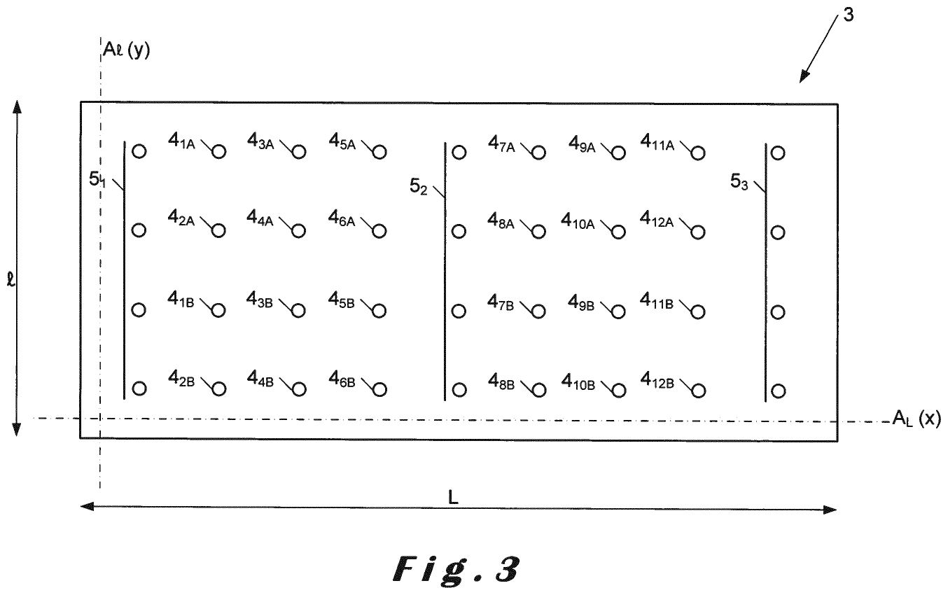

FIG. 3 illustrates in detail, the recovery system 3 according to the invention on which the recovery locations 4 and 5 are arranged according to a two-dimensional matrix arrangement in the form of points having a defined diameter, each of the points being separated by a minimum distance. The two-dimensional matrix arrangement is defined according to a system of coordinates (X; Y) which has a first coordinate X defined on a longitudinal axis (A_{L}) of a length (L) of said recovery system 3 and a second coordinate Y defined on a longitudinal axis (Aₗ) of a width (l) of said recovery system 3. According to a preferred embodiment, the recovery system 3 comprises at least 12 separate recovery locations (4₁-4₁₂) intended to respectively, simultaneously and specifically detect and/or quantify at least 12 analytes of separate classes present in a sample E and at least three recovery locations 5 intended for a control of the detection threshold or being used as a calibrator. Furthermore, each of the recovery locations (4₁-4₁₂ and 5₁-5₃) is arranged in two samples (4_{1A}; 4_{1B}-4_{12A}; 4_{12B}).

It is understood that the present invention is in no way limited to the embodiments described above and that modifications can be applied to them without moving away from the scope of the appended claims.

### EMBODIMENTS ACCORDING TO THE INVENTION—EXAMPLES

### Example 1: Example of Composition of a Buffer for the Reaction Mixture and Example of a Method for Preparing the Reaction Mixture

TABLE 1

| Salts and additives | Final concentration (nM) |
|---|---|
| TRIS | 20-25 |
| HEPES | 3-10 |
| NaCl | 4-8 |
| MgCl2 | 0-2 |
| Sugar | 50-100 |
| BSA | 0-1 |
| Glycerol | 10-30 |
| Tween | 0-1 |

To this buffer are added recognition molecules and/or competitive ligands. After incubating the mixture for one night at 4° C., this is lyophilised. During the carrying out of the test, 250 μl of sample to be tested will be added to the reaction mixture thus obtained.

### Example 2: Example of Coupling Recognition Molecules to Fluorophore Rhodamine B

“Beta” and “Tetra” receptors and DNA oligonucleotides are obtained according to the method described in EP1712914A1.

Monoclonal antibodies are purified on the protein A or protein G column according to the species and of the isotype. The antibodies are then stored at −20° C. in the phosphate buffer 10 mM NaCl 140 mM pH7.4.

The rhodamine B used has a N-hydroxysuccinimidyl(NHS)-esters residue which has the particularity of reacting with the amine groups of proteins with a basic pH.

The recognition molecules (antibodies and/or receptors) are dialysed for one night in a carbonate buffer 50 mM pH 8.5.

The fluorophore is dissolved in DMF at 5 mg/ml.

The recognition molecule and the fluorophore (the visualisation molecule) are brought together in a molar ratio of around ¼ for one hour away from light.

Finally, the chemical reaction is stopped during the complex dialysis with a phosphate buffer 10 mM pH 7.4.

Other types of chemical bonds can be achieved, with fluorochromes having a maleimide or carboxyl group.

Other types of fluorophores can be used, such as FITC, Alexa, DyLight, etc.

The coupling of the recognition molecules can also be carried out with colorimetric nanoparticles (gold, latex, carbon nanoparticles, etc.), as much by covalent coupling, as by electrostatic adsorption.

### Example 3: Example of Composition of the Reaction Mixture and Example of Recovery Elements Bonded on the Recovery System

TABLE 2

| | Molecules of | Ligands bonded | | | |
|---|---|---|---|---|---|
| | the reaction | on the recovery | | | Analytes |
| Channels | mixture | system | Class | Family | detected |
| CTL1 | Control | Control | control | control | / |
| | antibody 1 | antigen 1 | | | |
| BETA | Beta receptor | βlactams | βlactams | antibiotics | 27 |
| CEFA | Anti-cefalexin | cefalexin | βlactams | antibiotics | 2 |
| | monoclonal | | | | |
| | antibody | | | | |
| TETRA | Tetra receptor | DNA | tetracyclines | antibiotics | 10 |
| | | oligonucleotides | | | |
| SULFA | Anti- | sulphonamides | sulphonamides | antibiotics | 20 |
| | sulphonamide | | | | |
| | antibody | | | | |
| SDX | Anti- | sulphadoxine | sulphonamides | antibiotics | 1 |
| | sulphadoxine | | | | |
| | antibody | | | | |
| QUINO | Anti- | fluoroquinolones | fluoroquinolones | antibacterial | 20 |
| | fluoroquinolone | | | agents | |
| | antibodies | | | | |
| CAP | Anti- | chloramphenicol | phenicols | antibiotics | 1 |
| | chloramphenicol | | | | |
| | antibody | | | | |
| MELA | Anti-melamine | melamine | melamine | adulterant | 4 |
| | antibody | | | | |
| AFLA | Anti- | aflatoxineM1 | mycotoxins | toxins | 2 |
| | aflaxotineM1 | | | | |
| | antibody | | | | |
| CTL2 | Control | Control antigen 2 | control | control | / |
| | antibody 2 | | | | |
| COLI | Anti-colistin | colistin | polymyxins | antibiotics | 1 |
| | antibody | | | | |
| NEO | Anti-neomycin | neomycin | aminoglycosides | antibiotics | 2 |
| | antibody | | | | |
| GEN1 | Anti-gentamicin | gentamicin | aminoglycosides | antibiotics | 2 |
| | antibody | | | | |
| STR | Anti- | streptomycin | aminoglycosides | antibiotics | 2 |
| | streptomycin | | | | |
| | antibody | | | | |
| TYLO | Anti-tylosin | tylosin | macrolides | antibiotics | 2 |
| | antibody | | | | |
| LINCO | Anti- | lincosamides | sulphonamides | antibiotics | 3 |
| | lincosamide | | | | |
| | antibody | | | | |
| SPIRA | Anti-spyramicin | spyramicin | macrolides | antibiotics | 2 |
| | antibody | | | | |
| ERY | Anti- | erythromycin | macrolides | antibiotics | 3 |
| | erythromycin | | | | |
| | antibody | | | | |
| CTL1 | Control | Control antigen 3 | control | control | / |
| | antibody 3 | | | | |

| | |
|---|---|
| TOTAL | 104 analytes detected and distinguished via 17 channels |

### Example 4: Example of Carrying Out the Test and Results Obtained

A milk sample is put into contact with the reaction mixture (comprising the buffer and the recognition molecules and/or the competitive ligands in lyophilised form) for 3 minutes at 30° C. Then, the upstream end of the migration direction of the recovery system is immersed in the solution (comprising the sample and the reaction mixture). After an incubation of 10 minutes at 30° C., the reading of the results is carried out using an optical device.

The results are outlined in table 3.

TABLE 3

| | Concentrations | | | |
|---|---|---|---|---|
| | targeted by the | | Signal | |
| | test | Concentration | (arbitrary | Instrumental |
| Channels | (ppb; μg/kg) | (ppb; μg/kg) | unit) | interpretation |
| BETA | ≥4 | 2 | 1.04 | negative |
| | | 4 | 0.68 | positive |
| CEFA | ≥2 | 1 | 1.09 | negative |
| | | 2 | 0.64 | positive |
| TETRA | ≥50 | 30 | 1.10 | negative |
| | | 50 | 0.71 | positive |
| SULFA | ≥100 | 50 | 1.08 | negative |
| | | 100 | 0.71 | positive |
| SDX | ≥100 | 50 | 1.08 | negative |
| | | 100 | 0.69 | positive |
| QUINO | ≥20 | 10 | 1.29 | negative |
| | | 20 | 0.69 | positive |
| CAP | ≥0.3 | 0,2 | 1.01 | negative |
| | | 0,3 | 0.84 | positive |
| MELA | ≥15 | 10 | 1.11 | negative |
| | | 15 | 0.86 | positive |
| AFLA | ≥0.3 | 0,1 | 1.05 | negative |
| | | 0,3 | 0.93 | positive |
| COLI | ≥25 | 20 | 1.14 | negative |
| | | 25 | 0.72 | positive |
| NEO | ≥1200 | 900 | 1.04 | negative |
| | | 1200 | 0.69 | positive |
| GEN | ≥80 | 60 | 1.03 | negative |
| | | 80 | 0.68 | positive |
| STR | ≥200 | 150 | 1.05 | negative |
| | | 200 | 0.68 | positive |
| TYLO | ≥40 | 30 | 1.14 | negative |
| | | 40 | 0.80 | positive |
| LINCO | ≥80 | 60 | 1.08 | negative |
| | | 80 | 0.66 | positive |
| SPIRA | ≥50 | 30 | 1.05 | negative |
| | | 50 | 0.79 | positive |
| ERY | ≥20 | 10 | 1.23 | negative |
| | | 20 | 0.73 | positive |

1. A diagnosis kit for simultaneously and specifically detecting analytes present in a liquid sample, the diagnosis kit comprising an immuno-chromatographic diagnosis system, the immuno-chromatographic diagnosis system comprising:
(i) at least one reaction mixture comprising recognition biological molecules and/or competitive ligands labeled with at least one visualization molecule detectable with fluorescence; and
(ii) at least one recovery system comprising a solid support to which are bonded other competitive ligands and/or recognition biological molecules, at distinct and known recovery locations, which are arranged according to a two-dimensional matrix arrangement to enable identification of analytes present in the liquid sample based on localization of recovery locations on the solid support;
wherein:
the two-dimensional matrix arrangement is defined according to a system of coordinates comprising a first coordinate (X) and a second coordinate (Y), wherein for a given coordinate X, a plurality of recovery locations along Y corresponds to a first plurality of different recognition biological molecules and/or competitive ligands; and for a given coordinate Y, a plurality of recovery locations along X corresponds to a second plurality of different recognition biological molecules and/or competitive ligands, wherein distinct recovery locations on the solid support correspond to detection of distinct analytes in the liquid sample;
a diagnosis couple of the immuno-chromatographic diagnosis system, for detection of a given analyte, consists of a competitive ligand and a recognition biological molecule, wherein the recognition biological molecule is disposed in the at least one reaction mixture and the competitive ligand is bonded to a given recovery location, or vice versa; and
a reacted liquid sample is produced by an interaction of the at least one reaction mixture with the liquid sample that occurs in a container that is separate from the recovery system, wherein the reacted liquid sample is configured for contact with the solid support and the recovery locations for immuno-chromatographic analysis of the reacted liquid sample; and
(iii) an optical device configured for optically reading at least part of the solid support, the optical device comprising:
a placement configured to receive the at least part of the solid support;
an optical unit configured to analyze the solid support, wherein the optical unit comprises:
a first light source configured to emit a first light beam to the placement according to an emission intensity and a first wavelength range;
an imaging system comprising an optical detector configured to provide an image of a visualization zone, wherein the visualization zone comprises at least a portion of the placement; and
a filter positioned between the placement and the imaging system and configured to filter a defined wavelength range; and
a communication system configured to:
obtain an item of information relative to the solid support;
a selection system configured to:
select, from a list of predefined analytes corresponding to recovery locations of the solid support, a selection of analytes to be detected and/or quantified in the liquid sample from the solid support;
an image processing system configured to:
process an image of the visualization zone to determine, from the item of information relative to the solid support, a finite number of subassemblies of the image, wherein each subassembly corresponds to a particular analyte; and provide data related to light intensities coming from the subassemblies;
a determination system configured to:
calculate, for each subassembly that corresponds to an analyte of the selection of analytes, a subassembly intensity; and
determine, based on the subassembly intensity, analyte information related to the liquid sample for each subassembly that corresponds to an analyte of the selection of analytes; and
a transmission system configured to transmit analyte information for each subassembly that corresponds to an analyte of the selection of analytes of the liquid sample.

2. The diagnosis kit according to claim 1, wherein the recovery locations are arranged according to a two-dimensional matrix arrangement in the form of points, each having a diameter of between 20 μm to 2 mm, between 100 to 500 μm, or between 250 and 400 μm.

3. The diagnosis kit according to claim 1, wherein the at least one recovery system comprises at least 5 distinct recovery locations that respectively, simultaneously, and specifically detect and/or quantify at least 5 distinct analytes present in the liquid sample, and at least one recovery location configured as a control and/or calibrator location.

4. The diagnosis kit according to claim 1, wherein the solid support comprises a membrane or a set of membranes.

5. The diagnosis kit according to claim 1, wherein the at least one visualization molecule detectable with fluorescence are fused to the recognition biological molecules and/or to the competitive ligands via a chemical and/or genetic coupling.

6. The diagnosis kit according to claim 5, wherein the chemical and/or genetic coupling is performed via at least one electrostatic force, at least one peptide bond, at least one reporter gene, or any combination thereof.

7. The diagnosis kit according to claim 1, wherein the analytes are selected from the group consisting of: drug residues, toxins, viruses, bacteria, hormones, heavy metals, adulterants, allergens, a mixture thereof, and any combination thereof.

8. The diagnosis kit according to claim 7, wherein the analytes comprise drug residues and are selected from the group consisting of: penicillins, cephalosporines, tetracyclines, sulphonamides, aminoglycosides, aminocyclitols, macrolides, quinolones, ionophores, carbadox, nitrofuran antibiotics, phenicols, a mixture thereof, and any combination thereof.

9. The diagnosis kit according to claim 1, wherein the optical device further comprises:
means configured for depiction and/or use of a selection profile, wherein the means enables selection of one or more analytes based on the selection profile.

10. A method for simultaneously and specifically detecting a plurality of analytes present in a liquid sample with a diagnosis kit according to claim 1, the method comprising:
contacting the at least one reaction mixture of the diagnosis kit comprising the recognition biological molecules and/or competitive ligands labeled with at least one visualization molecule detectable with fluorescence with the liquid sample to obtain a reacted liquid sample;
incubating the reacted liquid sample, at a temperature of between 0 and 70° C., for a duration less than or equal to 15 minutes;
soaking an end of the at least one recovery system of the diagnosis kit that comprises at least part of the solid support in the reacted liquid sample;
incubating the end of the at least one recovery system of the diagnosis kit within the reacted liquid sample, at a temperature of between 0 and 70° C., for a duration less than or equal to 15 minutes; and
qualitatively interpreting a result with the optical device of the diagnosis kit.

11. The method of claim 10, the at least one recovery system of the diagnosis kit comprises distinct recovery locations, and wherein the recovery locations are arranged according to a two-dimensional matrix arrangement in the form of points, each having a diameter of between 20 μm to 2 mm, between 100 to 500 μm, or between 250 and 400 μm.

12. The method according to claim 10, wherein the at least one recovery system of the diagnosis kit comprises at least five distinct recovery locations that respectively, simultaneously, and specifically detect and/or quantify at least 5 distinct analytes present in the liquid sample, and wherein at least one recovery location is configured as a control and/or calibrator location.

13. The diagnosis kit according to claim 3, wherein the optical device further comprises:
means configured for depiction and/or use of a selection profile, wherein the means enables selection of one or more analytes based on the selection profile.

14. The method of claim 10, wherein the solid support of the diagnosis kit comprises a membrane or a set of membranes.

15. The method of claim 10, wherein the at least one visualization molecule detectable with fluorescence is fused to the recognition biological molecules and/or to the competitive ligands via a chemical and/or genetic coupling.

16. The method of claim 15, wherein the chemical and/or genetic coupling is performed via at least one electrostatic force, at least one peptide bond, at least one reporter gene, or any combination thereof.

17. The method of claim 10, wherein the plurality of analytes is selected from the group consisting of: drug residues, toxins, viruses, bacteria, hormones, heavy metals, adulterants, allergens, a mixture thereof, and any combination thereof.

18. The method of claim 17, wherein the plurality of analytes comprises drug residues and are selected from the group consisting of: penicillins, cephalosporines, tetracyclines, sulphonamides, aminoglycosides, aminocyclitols, macrolides, quinolones, ionophores, carbadox, nitrofuran antibiotics, phenicols, a mixture thereof, and any combination thereof.

19. The method of claim 10, wherein the optical device of the diagnosis kit further comprises: means configured for depiction and/or use of a selection profile, wherein the means enables selection of one or more analytes based on the selection profile.

20. The diagnosis kit according to claim 7, wherein the optical device further comprises:
means configured for depiction and/or use of a selection profile, wherein the means enables selection of one or more analytes based on the selection profile.
